(19) United States
(12) Patent Application Publication
(10) Pub. No.: US 2016/0128712 A1
(43) Pub. Date: May 12, 2016

(54) Double-Bladed Scalpel

(71) Applicant: Robert A. Ruggiero, SR., West Chester, PA (US)

(72) Inventor: Robert A. Ruggiero, SR., West Chester, PA (US)

(21) Appl. No.: 14/538,702

(22) Filed: Nov. 11, 2014

(51) Int. Cl. A61B 17/3211

(52) U.S. Cl. CPC A61B 17/3211

(57) ABSTRACT

Example ways of forming double-bladed scalpels and constituent parts are described. In one aspect a sleeve for coupling two-conventional scalpels together to form a double-bladed scalpel is described. Also described are male and female fastening mechanisms disposed along the sides of two independent scalpels. Each scalpel may be used independently of the other, or mated together via the fasteners to form a single double-bladed scalpel. In another aspect, a bracket is used to form a double-bladed scalpel from two-conventional scalpels. In another example, a double-bladed scalpel with a single handle is described.

### BACKGROUND

A scar resulting from an incision made by a surgeon in a first operation is usually minor if the boundaries of the skin along opposite sides of the incision have been properly closed following surgery. Usually, there are no differences in contour between the edges of the skin along the line of the incision, because they were produced at the same time by a single cutting device.

However, often it is necessary to perform additional operations at substantially the same site of the scar, such as for a Cesarean section. Put differently, one or more additional incisions are made along the line of the original incision, resulting in the formation of scar tissue. And with each repetition of the incision the width and height of the scar tissue increases, causing the scar to become unsightly.

Conventional operational procedure adopted for the removal of such scar tissue is to employ a single conventional scalpel and to cut through the skin along lines respectively disposed at opposite sides of the scar tissue. The skin between the corresponding ends of such lines is cut through and then the skin between the lines is then removed.

This procedure is often difficult and unsatisfactory because the skin immediately following the cut made by the scalpel pulls away irregularly, there being scar tissue along one side of the incision and virgin tissue along the other side, and after the scar tissue is removed the edges of the skin to be drawn together by sutures are not complementary in linear contour nor are they straight.

For this reason, an unsightly scar may be unavoidable and the difficulty of properly suturing or uniting the irregular edges of the virgin skin is time consuming and difficult.

Surgeons have, from time-to-time, tried to overcome the above difficulties attendant upon removing scar tissue by attempting to hold a pair of scalpels in one hand and to make the incision along parallel paths at the same time.

Obviously this is a difficult procedure and a hazardous one. The points of the blades of the scalpels should be positioned and held to cut the same depth at the same time. To attempt to hold a pair of separate scalpels is awkward at best, and there is imminent danger of slippage of one or the other with unacceptable risks to the patient.

Double-bladed scalpels have been developed to enable a physician to make two spaced-apart-parallel incisions simultaneously. These scalpels usually contained two blades in fixed relative position to each other for producing strips of one fixed width. Other scalpels were developed in which the distance of separation between the cutting edges of the blades were adjustable manually for making incisions of different widths.

Unfortunately, very few of these scalpels were commercially developed nor adopted by surgeons in the field. As a result, most surgeons are faced with the same problems encountered by surgeons over several decades when attempting to cut through scar tissue. That is, most surgeons today use a single scalpel to make incisions along scar tissue, and are no better equipped to remove scar tissue than over 50 years ago.

### SUMMARY

Example double-bladed scalpels and constituent parts are described. In one example, a sleeve for creating a single unitary double-bladed scalpel from two conventional independent scalpels is disclosed.

Also described is a doubled-bladed scalpel with a single dedicated handle. The handle may include an integrated sleeve.

Also described, are scalpels containing fastening mechanisms disposed thereon and/or therein for coupling two scalpels together to form a single unitary double-bladed scalpel. The fastening mechanisms allow a surgeon to use each scalpel as a single unit when not fastened to another scalpel.

In one aspect, a pair of conventional scalpels are joined side-by-side by an elastomeric-tubular sleeve. The sleeve is adapted to slide over the two conventional scalpels. When the sleeve is disposed over the housing of the scalpels, they are held in place in fixed relation to each other. That is, the sleeve secures the two scalpels in side-by-side alignment and in fixed-parallel relation with each other. The sleeve prevents the blades of each scalpel from independently moving with respect to the other. An inner surface of the sleeve may have a geometric shape contoured to fit snuggly and coextensively around the housings of two scalpels. The inner surface of the sleeve may be smooth or include a gripping pattern. The inner surface of the sleeve may also be lubricated with silicone or other material to permit the sleeve to be slid over the housings of the scalpels without too much resistance. However, friction generated by shear forces between the inner surface of the sleeve, and outer surfaces of the housings for the scalpels, retains the sleeve in place after it is slid in into place over the scalpels.

The sleeve may have varying longitudinal lengths sufficient to hold and join two scalpels together in fixed relation to each other. Typically, the distal end of the sleeve is aligned (or in close proximity thereto) with a distal end, i.e., front/blade end, of each scalpel's housing. In one example, the sleeve is about an inch in length, but may be shorter or longer as appreciated by those skilled in the art after having the benefit of this disclosure.

An outer surface of the sleeve comprises one or more surface patterns and/or gripping elements that permits the surgeon to tactilely grip, and readily feel the contours of the surface pattern/gripping elements through the surgeon's gloves. As appreciated by those skilled in the art after having the benefit of this disclosure, the surface pattern may improve stability and minimize slippage during use of the scalpels.

In one embodiment, the surface pattern includes a transverse lip that may extend across the width of the sleeve at a location toward a distal end of the sleeve. The transverse lip serves as a guard for preventing the surgeon's fingers from accidently sliding forward toward the blade area when exerting downward pressure on the scalpels during surgery. The transverse lip may be tubular in shape, and be disposed around the sleeve.

In one embodiment, the surface pattern may also include a center protrusion on a facing surface of the sleeve. The center protrusion may be further located along a center-longitudinal axis of the sleeve when the sleeve is slid over the scalpels. The center protrusion may be elevated above other protrusions on the surface pattern, and/or be of a pattern type, and/or length, which allows the surgeon to readily detect where the center line of the joined scalpels reside. In other words, the center protrusion is generally aligned with a center-longitudinal axis of both scalpels when joined together by the sleeve.

In one embodiment, the sleeve may include varying geometric contours, such as an hour-glass shape, or may include rings or other geometric shapes for enhanced gripping. If the sleeve includes an hour-glass shape, the aforementioned transverse lip may be replaced by an upper-most ramp (i.e. flared portion) of the hour-glass shaped sleeve.

In another embodiment, the sleeve includes a fastening device to permit the sleeve to be wrapped around the housings of two-paired scalpels instead of slid over the scalpels. The fastening device may include a clasp, or hook and clasp fasteners (i.e., Velcro®).

In another embodiment, the sleeve and/or the housing of each scalpel include suitable interconnection devices to permit the scalpels to be snapped together in a fixed relationship to each other. For example, a first scalpel may include one or more female apertures defined on a first side of the housing. A second scalpel may include male members defined on a second side of the housing opposite the female apertures for insertion therein.

In one example, the sleeve may include a clip to secure the scalpels side-by-side. In this example, the sleeve may or may not include an upper surface or gripping features as well as the transverse lip.

In yet another embodiment, the width between each cutting blade of each scalpel may be adjusted before the sleeve is fastened around the scalpels. For instance for scars that are narrow (i.e., approximately 2 mm in width), it may be possible to simply join the two scalpels together via the sleeve. But for wider scars (i.e., 4 mm), a small grommet or other material may be sandwiched between the two inner portions of each housing of both scalpels to widen the distance between the blades of each scalpel, before the sleeve is slid over or wrapped around the scalpels.

In another implementation, the sleeve may form a permanent-integrated grip of a single-handle-double-bladed scalpel. That is, the scalpel includes only one handle (instead of two conventional handles secured together) having two parallel blades extending from a distal end of the handle. In this example, the width of between the blades of the scalpel may be preconfigured at different widths ranging in size from about 2 mm to 5 mm.

This summary is provided to introduce a selection of concepts in a simplified form that are further described below. This summary is not necessarily intended to identify key features or essential features of the claimed subject matter, nor is it necessarily intended to be used as an aid in determining the scope of the claimed subject matter.

The foregoing outlines examples of this disclosure so that those skilled in the relevant art may better understand the detailed description that follows. Additional embodiments and details will be described hereinafter. Those skilled in the relevant art should appreciate that they can readily use any of these disclosed embodiments as a basis for designing or modifying other structures or functions for carrying out the invention, without departing from the spirit and scope of the invention.

Reference herein to “one embodiment”, “an embodiment”, “an implementation” “an example” or similar formulations herein, means that a particular feature, structure, operation, or characteristic described in connection with the embodiment, is included in at least one embodiment of the present invention. Thus, different appearances of such phrases or formulations herein do not necessarily refer to the same embodiment. Furthermore, various particular features, structures, operations, or characteristics may be combined in any suitable manner in one or more embodiments.

### BRIEF DESCRIPTION OF THE DRAWINGS

The detailed description is described with reference to the accompanying figures. In the figures, the left-most digit(s) of a reference number identifies the figure in which the reference number first appears. The figures are not necessarily drawn to scale.

FIG. 1 is a perspective view of two conventional scalpels coupled together by an elastomeric-tubular sleeve thereby forming a double-bladed scalpel.

FIG. 2 is an end view of the sleeve shown in FIG. 1.

FIG. 3 is a side view of the scalpels and sleeve shown in FIG. 1

FIG. 4 is a top view of the sleeve shown in FIGS. 1-3.

FIG. 5 is a side view of an hour-glass shaped sleeve.

FIG. 6 is an end view of bracket for forming a double-bladed scalpel.

FIG. 7 is a perspective view of a double-bladed scalpel using the bracket shown in FIG. 6.

FIG. 8 is a perspective view of two scalpels having complementary fasteners disposed on at least one side wall of each scalpels.

FIG. 9. is a perspective view of the two scalpels (shown in FIG. 8) coupled together.

FIG. 10 is a perspective view of a double-bladed scalpel having a single handle.

### DETAILED DESCRIPTION

### Example Sleeve

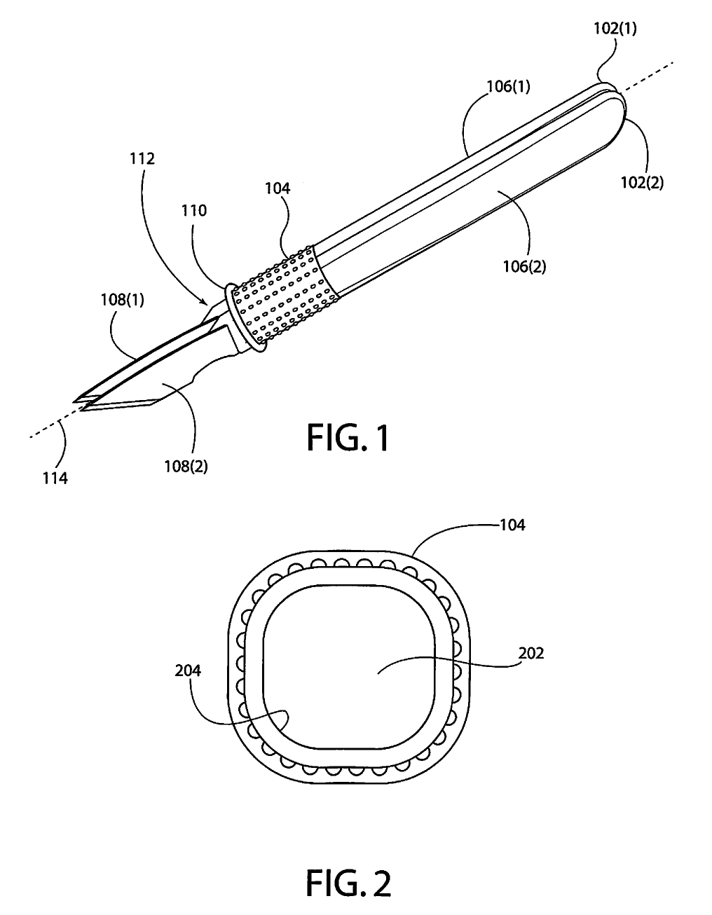

FIG. 1 shows a perspective view of two conventional scalpels 102(1) and 102(2) joined side-by-side via an elastomeric-tubular sleeve 104. Each scalpel (referred to hereinafter generally as reference number 102) is a convention scalpel such as BD Bard-Parker™ Protected Disposal Scalpels™ or any other suitable scalpel.

Sleeve 104 is adapted to fit around or slide over housings (i.e., the handles) (hereinafter generally referred to as reference number 106) of scalpels 102. Sleeve 104 is positioned near blades 108(1), 108(2). Sleeve 104 secures scalpels 102 (FIG. 1) in side-by-side alignment and in fixed-parallel relation with each other. That is, sleeve 104 when disposed around scalpels 102 is expanded to fit tightly around housings 106, thereby keeping each scalpel from independently moving with respect to each other.

Sleeve 104 may be constructed of any suitable elastomeric material that is strong and rigid enough to frictionally engage and hold both scalpels in fixed relation to each other. For instance, sleeve 104 may be constructed of a resilient material, such as thermoplastic elastomer, rubber, vinyl, polyester, resilient plastic, a silicone material, a composite material, or any other suitable materials for the purpose of securing two scalpels as well as to provide a stable and comfortable grip to a surgeon.

FIG. 2 shows a cross-sectional view of sleeve 104. Referring to FIG. 2, an inner surface 204 of sleeve 104 may have a geometric shape contoured to fit snuggly and coextensively around the housings of two scalpels. Inner surface 204 of the sleeve may be smooth or include a gripping pattern. Inner surface 204 may also be lubricated with silicone or other material to permit the sleeve to be slid over housings 106 of scalpels 102 without too much resistance. However, friction generated by shear forces between inner surface 204 of sleeve 104, and outer surfaces of housings 106, retains sleeve 104 in place after it is slid into place over the scalpels 102 (FIG. 1).

As depicted in FIG. 2, hole 202 of sleeve 104 is rectangular, which is commensurate in shape with the outer shape of two scalpels 102 in FIG. 1, but may be of other sizes and shapes that are commensurate in size and shape of the scalpels in which they are configured. In other words, the shape of hole 202 (see FIG. 2) of each sleeve 104 may be configured to match the housing shape and contour configurations of the outer surfaces of two scalpels, when aligned together, and abutting each other side-by-side.

Referring back to FIG. 1, sleeve 104 may have varying longitudinal lengths sufficient to hold and join two scalpels together in fixed relation to each other. Typically, distal end 110 of sleeve 104 is aligned (or in close proximity thereto) with a distal end 112, i.e., front end, of each scalpel's 102 housing 106. In one example, the sleeve 104 is about an inch in length, but may be shorter or longer as appreciated by those skilled in the art after having the benefit of this disclosure.

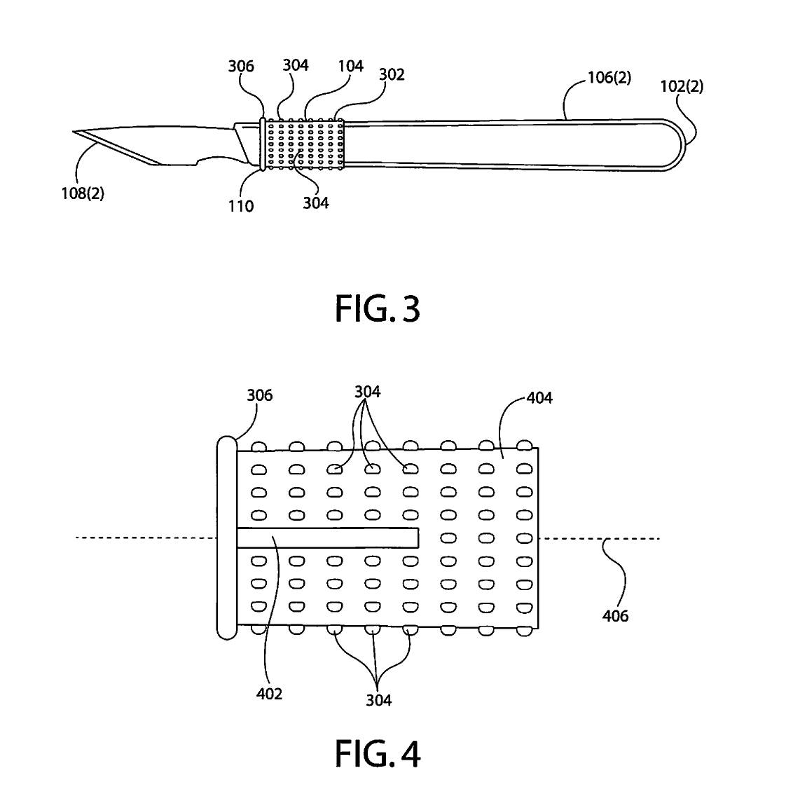

The outer gripping surface of sleeve 104 is depicted in FIGS. 3 and 4. For instance, FIG. 3 shows a side view of the double-bladed scalpel arrangement shown in FIG. 1. Here, outer surface 302 of sleeve 104 includes one or more gripping elements 304 extending longitudinally away from surface 302. In the example depicted in FIG. 3, gripping elements 304 include a plurality of cylindrical-geometric shapes extending from surface 302. However, as appreciated by those skilled in the art, after having the benefit of this disclosure, gripping elements 304 may be constructed of any myriad of different shapes, and patterns.

Gripping elements 304 allow a surgeon to comfortably grip double-bladed scalpels 102 while minimizing slippage during use of the scalpels. In some implementations, a surgeon may discern the contours of gripping elements 304 through surgical gloves. In one example each gripping element 304 adjacent gripping elements are spaced apart at least ⅛ inch from each other, and longitudinally extend at least ¼ inch away from surface 302. Each gripping element 304 is compressible by an average female squeezing the gripping elements 304.

In one embodiment, surface pattern 304 also includes a transverse lip 306 that may extend across the width of sleeve 104 at a location toward a distal end 110 of sleeve 104. Transverse lip 306 serves as a guard for preventing the surgeon's fingers from accidently sliding forward toward the blade area when exerting downward pressure on the scalpels during surgery. Transverse lip 306 may be tubular in shape or a plurality of other geometric shapes (rounded, pointed, square, etc.), and may be disposed circumferentially around sleeve 104. As appreciated by those skilled in the art, an indentation (i.e., a groove) (not shown) may also serve as a finger guard in lieu of, or in conjunction with lip 306.

Referring to FIG. 4, is top view of sleeve 104. As shown, surface pattern 304 may also include a center protrusion 402 on a facing surface 404 (i.e. top surface) of sleeve 104. Center protrusion 402 is located along a center-longitudinal axis 406 of sleeve 104, when sleeve 104 is disposed over scalpels 102. Center protrusion 402 may be elevated above other patterns on surface pattern 304, and/or may be of a pattern type, and/or length, which allows the surgeon to readily detect where the center line of a double-bladed scalpel resides. In other words, center protrusion 402 is generally aligned with a center-longitudinal axis 114 (see FIG. 1) of scalpels 102 when joined together by sleeve 104 (FIG. 1).

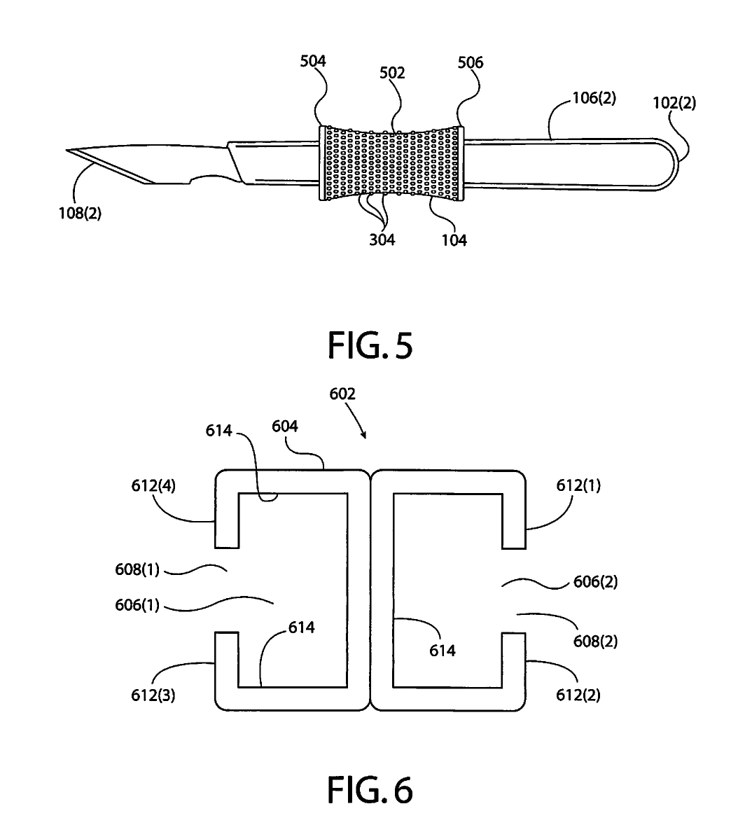

Sleeve 104 may include varying geometric contours. For instance, as depicted in FIG. 5, sleeve 104 is hour-glass shaped. A distal end (i.e., front end) of sleeve 104 nearest blades 108 includes a flared edge 504. Also, a proximal end (i.e., rear end) of sleeve 104 includes a flared edge 506. In the illustrated embodiment, flared edge 504 serves as a finger guard in lieu of transverse lip 306 (FIG. 3).

### Example Holder Clip

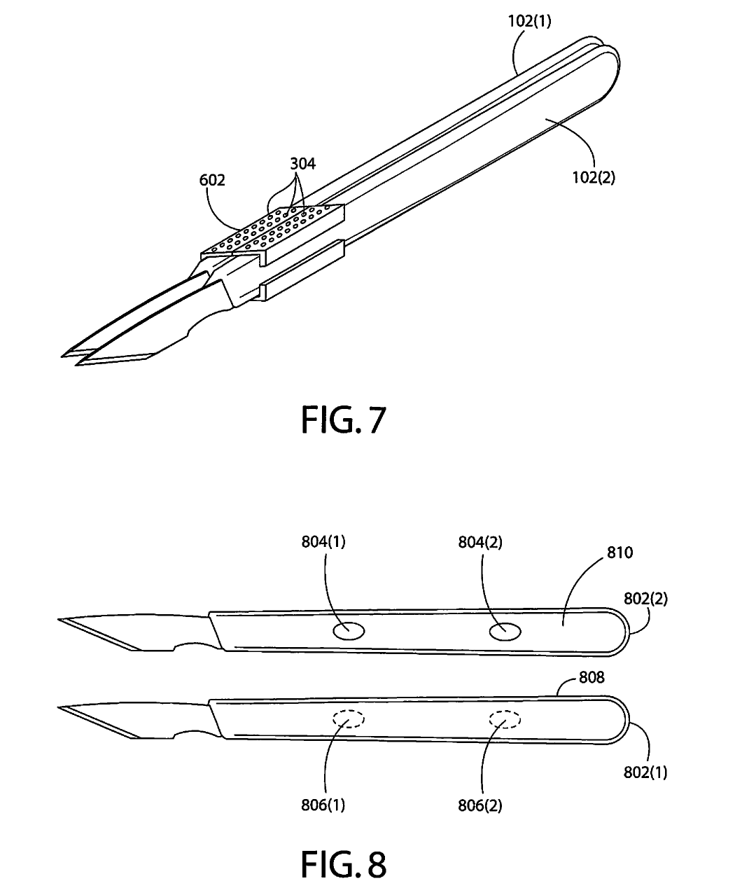

A holder may be used to secure two scalpels in side-by-side relation forming a double-bladed scalpel. As best illustrated in FIG. 6, is a perspective view of holder assembly 602 for securing two scalpels side-by-side. Holder assembly 602 includes a semi-rigid body 604 having side-by-side chambers 606(1), 606(2), which are configured to securely hold scalpels 102(1), 102(2) (see FIG. 7) therein. That is, each chamber 606(1), 606(2) is configured to fit around a housing 106(1), 106(2), respectively, of each scalpel 102.

Holder assembly 602 allows a surgeon to quickly and easily attach two conventional scalpels into chambers 606(1), 606(2). The exact dimensions and configurations of each chamber 606 are pre-molded, and selected to conform to the shape of scalpels selected by the surgeon.

Holder assembly 602 may be produced from semi-flexible, molded plastic. However, as appreciated by those skilled in the art after having the benefit of this disclosure, other materials with similar characteristics can be used in place of a plastic, such as rubber.

In one embodiment, each chamber is approximately the same dimension or slightly smaller than housings 106. Accordingly, each housing 106 may be slidably inserted into chamber 606 or removed therefrom. Alternatively, each chamber 606 may snap-fit over or around a housing 106 of a scalpel 102 when pressed into each chamber 606 via openings 608(1), 608(2). Because each chamber 606 is resilient and semi-rigid, it may flex and expand slightly to receive a housing 106 of scalpel 102 therein. One or more holding tabs 612(1), 612(2), 612(3), 614(4) in conjunction with inner wall(s) 615 of each chamber 606 retain and secure each scalpel 102 inside chambers 606. As appreciated by those skilled in the art, it may be possible use less than four holding tabs. In addition, there may be only a single center-inner wall 615 (i.e., a shared-center support wall) separating the first and second chambers from each other.

FIG. 7 shows a perspective view of holder assembly 602 including scalpels 102 secured therein. As shown in FIG. 7, holder assembly 602 may include surface elements 304 and finger guards in accordance with the principles described above with reference to sleeve 104 in FIGS. 1-4 above. Various other grip patterns or texturing may be used in alternatives.

### Example Fastening Assemblies

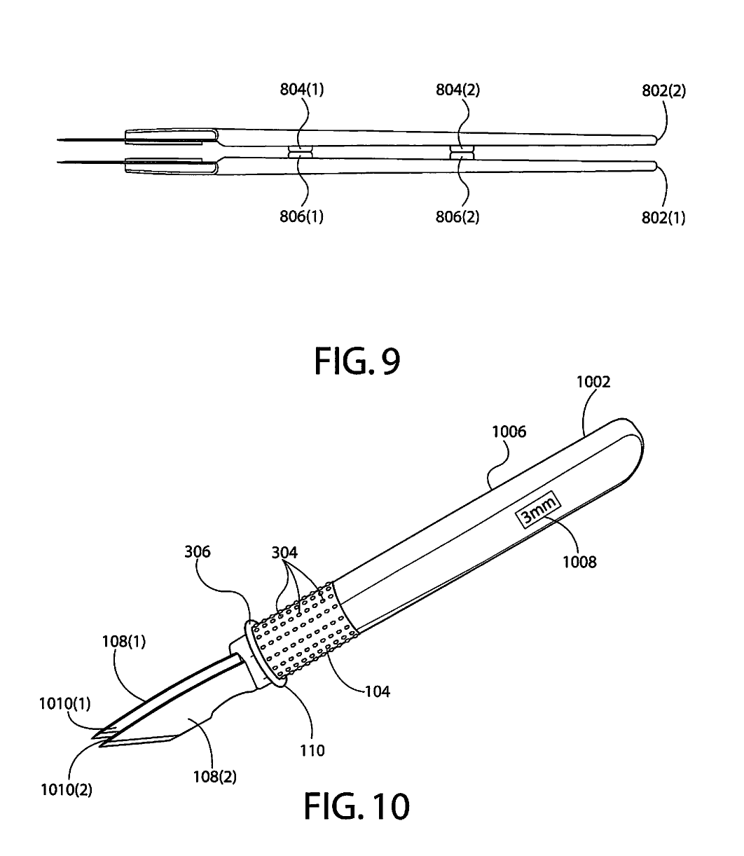

In another embodiment, a scalpel may also include male and female snap parts. For example, FIG. 8 shows a first scalpel 802(1), including male-snap parts 806(1), 806(2) disposed on a left-side wall 808, and second scalpel 802(2) including female-snap parts 804(1), 804(2), disposed on a right-side wall 810. To achieve a double-bladed scalpel, scalpel 802(1) is aligned side-by-side with scalpel 802(2) so that corresponding fasteners (female-snap parts 804(1), 804(2) are engaged with male-snap parts 806(1), 806(2), respectively). FIG. 9 shows a scalpels 802(1), 802(2) engaged and mated with each other via snap parts 804, 806.

Snaps parts 804, 806 may be any suitable snap fastener widely used in different industries such as batteries, clothes, and the like. The snaps may be comprised of metal or other suitable materials such as semi-flexibly plastic. Snap parts 804, 806 may be an integrated-molded feature of each scalpel 802 or may be attached thereto by glue or other fastening means.

In the depicted examples of FIGS. 8 and 9, snap pairs are positioned a suitable distance apart, such as close to the distal (i.e. blade end) and proximal ends (i.e. end furthest from the blades) of each handle. Although two fastening pairs are shown, it is also appreciated by those skilled in the art after having the benefit of this disclosure that more or less fasteners may be used to couple scalpels together.

In addition, although female/male snap parts are shown, it is also appreciated that other fastening mechanisms, such as brackets, clips, magnets, and related fasteners may be employed, in lieu of or in addition to snaps, along the side wall of each scalpel.

Still further, although female and male fastening mechanism are only shown on one side wall 808, 810 of each scalpel 802, it is possible that fastening mechanisms may be included on the right and left side walls of each scalpel to ensure there is always mating pair of scalpels in which to form a double-bladed scalpel.

In addition, a first scalpel may include one or more female apertures defined on a first side of the housing. A second scalpel may include male members defined on a second side of the housing opposite the female apertures for insertion therein. In one example, the interconnecting female and male system may be similar to lockable ball and socket joints in place of sockets. Thus, the terms male snap parts and female-snap parts, as used herein, are intended to include ball and socket fastening devices and their equivalents.

A double-bladed scalpel may also have an integrated sleeve 104. That is, as best illustrated in FIG. 10, a double-bladed scalpel 1002 includes two blades 108(1), 108(2), and a single handle 1006. Sleeve 104 is integral to handle 1006. That is, sleeve 104 is fixedly attached to handle 1006. Sleeve 104 may be co-molded with handle 606 or fixedly attached thereto by internal means (not shown). Thus, sleeve 104 may also form a permanent-integrated grip of a single-handle-double-bladed scalpel 1002.

Still referring to FIG. 10, the distance between the blades of the scalpel may be preconfigured at different widths ranging in size from about 2 mm to 5 mm. Indicia 1008 indicating a corresponding width size between inner edges 1010(1), 1010(2) may be marked directly on the handle 1006.

Blades 108(1), 108(2) are preferably fixedly mounted in handle 1006 in a permanent manner, such as for a single-use scalpel, but could also be removably attached to handle 1006. Handle 1006 is typically an integrally solid rigid material. For instance, handle 1006 may be formed integrally of solid rigid material, such as rigid (or semi-rigid) plastic resins, metal, or other suitable materials.

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

1-16. (canceled)

17. A holder for securing a first handle of first scalpel and a second handle of a second scalpel therein, the holder comprising:
a semi-rigid body having parallel side-by-side first and second chambers, which are symmetrical with respect to each other, wherein each chamber includes openings at each distal end of the chamber;
wherein the first chamber is rectangular in shape and includes four semi-rigid lateral walls arranged about an axis, and configured to receive and securely hold the first handle, of a rectangular cross section, inside the first chamber in a position coaxial with the first chamber,
wherein the second chamber is rectangular in shape and includes four semi-rigid lateral walls arranged about an axis, and configured to receive and securely hold the second handle, of a rectangular cross section, inside the second chamber in a position coaxial with the second chamber,
wherein the first and second chambers share a lateral wall, which forms a center-longitudinal axis of the holder;
wherein the first and second chambers are also configured to receive and securely hold the first and second scalpels, respectively, in fixed and transversely parallel side-by-side positions relative to each other; and
an outer surface of at least one of the walls of the chambers, the outer surface including one or more gripping elements extending longitudinally away from the outer surface; wherein at least one of the gripping elements is aligned with the center-longitudinal axis of the holder.

18. The holder of claim 17, in which the walls are constructed of plastic.

19. The holder of claim 17, wherein the at least one gripping element aligned with the center-longitudinal axis extends longitudinally a furthest distance away from the outer surface than any other of the one or more gripping elements.
